(19) United States
(12) Patent Application Publication
(10) Pub. No.: US 2016/0113895 A1
(43) Pub. Date: Apr. 28, 2016

(54) Novel Use of Adapalene in Treating Esophageal Cancer and Gastrointestinal Stromal Tumor

(71) Applicant: Biodelight Biotech Inc., Hsinchu County (TW)

(72) Inventor: Thy-Hou LIN, Hsinchu County (TW)

(73) Assignee: Biodelight Biotech Inc.

(21) Appl. No.: 14/852,996

(22) Filed: Sep. 14, 2015

Related U.S. Application Data
(63) Continuation-in-part of application No. 14/151,045, filed on Jan. 9, 2014, now abandoned.
(60) Provisional application No. 61/758,927, filed on Jan. 31, 2013.

(51) Int. Cl. A61K 31/192; A61K 9/08; A61K 9/00

(52) U.S. Cl. CPC A61K 31/192; A61K 9/0019; A61K 9/08

(57) ABSTRACT

A novel use of 6-[3-(1-adamantyl)-4-methoxyphenyl]-2-naphthalenecarboxylic acid or a pharmaceutically acceptable salt or functional derivative thereof in treating a cancer, and in particular esophageal cancer and gastrointestinal stromal tumor, is provided.

### CROSS REFERENCE TO RELATED APPLICATIONS

This patent application is a continuation-in-part application of U.S. patent application Ser. No. 14/151,045, filed Jan. 9, 2014, which claims the priority benefit of prior U.S. Provisional Patent Application Ser. No. 61/758,927, filed Jan. 31, 2013. The disclosure of U.S. patent application Ser. No. 14/151,045 is incorporated herein by reference.

### FIELD OF THE INVENTION

The present invention relates to a novel use of 6-[3-(1-adamantyl)-4-methoxyphenyl]-2-naphthalenecarboxylic acid or functional derivatives thereof in treating esophageal cancer and gastrointestinal stromal cancer.

### BACKGROUND OF THE INVENTION

High levels of expression of EGFR (epidermal growth factor receptor) are found in many human tumors, especially squamous carcinomas of the esophagus, intestine, lung, or head and neck. The constitutively active mutant EGFR has also been reported in gliomas, breast and lung tumors. This implies that activation of EGFR may derive the proliferation of these tumors and that the inhibitors of EGFR may be used as antitumor agents.

NSCLC (Non-small-cell lung carcinoma) is the most common form of lung cancer which counts for about 75% of the lung cancer cases in patients¹. In a significant fraction of patients it is caused by mutations in EGFR kinase domain. The substitution L858R in the activation loop or deletion of three to eight residues in the region spanning residues 746-759, extending from the β3 strand to the αC helix in the N-lobe of the kinase domain², are the two most frequently observed mutations. Although patients with this form of lung cancer can be successfully treated with marketed drugs such as Iressa® or Gefitinib and Tarceva® or Erlotinib, most of them develop resistance to these therapies within months of treatment³. In about half of these patients, the resistance is caused by a second mutation T790M of the gatekeeper residue of the kinase domain². There is an urgent need to discover targeted drugs for inhibiting the L858R+T790M and deletion+T790M mutant EGFR to treat these patients⁴. The L858R kinase domain has been shown to possess greater activity than the wild types. This is attributed to the fact that the mutation is able to lock the enzyme in a constitutively active conformation. However, a recent study has proposed that the mutation facilitates the dimerization of the receptor⁶. Both Iressa® or Gefitinib and Tarceva® or Erlotinib are ATP competitive inhibitors and exhibiting selectivity for the L858R mutant over wild type enzyme^{6,7}. This is reasoned that the ATP Km value of the mutant enzyme is higher than that of the wild type⁸. It has been suggested that the emergence of drug resistance in patients carrying the secondary mutation T790M is due to the fact that the ATP Km value of the double-mutant enzyme has been restored to that of the wild type level⁸.

Nsl-8 (or adapalene) is a retinoid compound originally approved by FDA for treating acne and keratosis. The chemical formula for nsl-8 is C₂₈H₂₈O₃ and the corresponding IUPAC name is 6-[3-(1-adamantyl)-4-methoxyphenyl]-2-naphthalenecarboxylic acid (CAS registry number: 106685-40-9). The chemical structure of nsl-8 is depicted as follows:

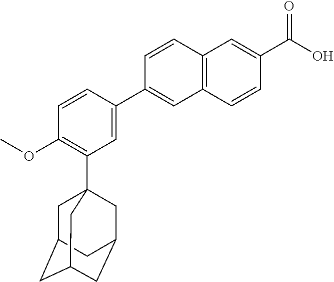 [SMILES: COc1ccc(-c2ccc3cc(C(=O)O)ccc3c2)cc1C12CC3CC(CC(C3)C1)C2]

### SUMMARY OF INVENTION

A primary objective of the present invention is to provide a use of 6-[3-(1-adamantyl)-4-methoxyphenyl]-2-naphthalenecarboxylic acid (or adapalene or nsl-8) or a pharmaceutical salt or derivative thereof in treating a cancer, as well as a pharmaceutical composition comprising adapalene or a pharmaceutical salt or derivative thereof as an active ingredient for treating a cancer.

Another objective of the present invention is to provide a method of treating a cancer in a subject comprising administering adapalene or a pharmaceutical salt or derivative thereof to the subject in need of such treatment.

Still another objective of the present invention is to provide a method of killing cancer cells in a subject comprising administering adapalene or a pharmaceutical salt or derivative thereof to the subject in need of such treatment.

A further objective of the present invention is to provide a use of adapalene or a pharmaceutical salt or derivative thereof in manufacturing a drug for treating a cancer.

In one aspect of the present invention, the subject of the invention is a mammal.

In another aspect of the present invention, the mammal is human.

In another aspect of the present invention, the adapalene or a pharmaceutical salt or derivative thereof is administered to a human at a daily dosage which is converted from a mouse daily dosage of about 40 mg/kg body weight.

Yet another aspect of the present invention, the subject in need of the treatment has developed resistance to Gefitinib or Erlotinib.

In one aspect of the present invention, the cancer is an EGFR associated tumor, and adapalene is an active inhibitor of EGFR.

In another aspect of the present invention, the cancer is associated with an elevated level of EGFR.

Yet another aspect of the present invention, the EGFR associated cancer is selected from esophageal cancer, gastrointestinal stromal tumor, lung cancer, breast cancer, head and neck cancer, pancreatic cancer, colorectal cancer or glioma cancer.

A further aspect of the present invention, the EGFR associated cancer is esophageal cancer, or gastrointestinal stromal tumor.

### BRIEF DESCRIPTION OF THE DRAWINGS

FIG. 1 shows 10 plots of enzymatic assay IC₅₀ results of nsl-1 to nsl-10.

FIGS. 2A, 2B, 2C and 2D show the 2D cell-based assays for measuring IC₅₀ for nsl-8 in four different NSCLC cell lines, A549, H1299, H460 and HCC827, respectively.

FIGS. 3A, 3B, 3C and 3D show the 3D cell-based assays for measuring IC₅₀ for nsl-8 in four different NSCLC cell lines, A549, H1299, H460 and HCC827, respectively.

FIG. 4 shows the growth of A549 implanted tumors in groups 1a and 1b animals. Test drug nsl-8 represented by squares was plotted versus vehicle control represented by triangles. Data are given as means+/−SEM.

FIG. 5 shows the changes of animal weights of A549 bearing animals of groups 1a and 1b. Test drug nsl-8 represented by squares was plotted versus vehicle control represented by triangles. Data are given as means+/−SEM.

FIG. 6 shows the primary tumor volumes (A549 model) measured in vivo for group 1a and 1b animals on day 69. Test drug nsl-8 (group 1b) versus vehicle control (group 1a) were plotted. Data are both given as means+/−SEM.

FIG. 7 shows the effect of nsl-8 on the growth of A549 implanted tumors in groups 2a, 2b, and 2c animals.

FIG. 8 shows the effect of nsl-8 on the growth of HCC827 implanted tumors in groups 3a, 3b, and 3c animals.

FIG. 9 shows the effect of nsl-8 on the body weight of A549 tumor-bearing mice.

FIG. 10 shows the effect of nsl-8 on the body weight of HCC827 tumor-bearing mice.

FIGS. 11A, 11B, 11C and 11D show the effect of nsl-8 on the blood cell counts of A549 tumor-bearing mice for total white blood cells (Total WBC), white blood cells (WBC), red blood cells (RBC) and platelets (PLT), respectively.

FIGS. 12A, 12B, 12C and 12D show the effect of nsl-8 on the blood cell counts of HCC827 tumor-bearing mice for total white blood cells (Total WBC), white blood cells (WBC), red blood cells (RBC) and platelets (PLT), respectively.

FIG. 13 shows the effect of nsl-8 on BUN, ALT, and AST determined for the A549 tumor-bearing mice.

FIG. 14 shows the effect of nsl-8 on BUN, ALT, and AST determined for the HCC827 tumor-bearing mice.

FIGS. 15A and 15B show docking Tarceva® or Erlotinib (FIG. 15A) or nsl-8 (FIG. 15B) into the active site of wild type EGFR kinase domain 2ity by ADDock.

FIGS. 16A and 16B show docking Tarceva® or Erlotinib (FIG. 16A) or nsl-8 (FIG. 16B) into the active site of mutant EGFR kinase domain 4i22 carrying double mutations L858R+T790M by ADDock.

### DETAILED DESCRIPTION OF THE INVENTION

### Pharmaceutical Composition of the Invention

The inventor of the present application intended to search some known drug databases to find a drug lead which might inhibit EGFR through binding with its kinase domain and thereby causing the cessation of growth for some tumors especially those of NSCLC. The theoretically searched drug lead has been studied experimentally through enzymatic and some 2D and 3D cell proliferation assays. It is surprised that nsl-8 (or adapalene) shows the activity of binding with the wild type EGFR kinase domain. Further, the antitumor efficacy of the searched drug lead nsl-8 (or adapalene) has been evaluated using some in vivo mouse xenograft models.

The pharmaceutical composition of the invention is provided by comprising 6-[3-(1-adamantyl)-4-methoxyphenyl]-2-naphthalenecarboxylic acid (or adapalene or nsl-8) or functional derivatives thereof as the active ingredient.

The functional derivatives of 6-[3-(1-adamantyl)-4-methoxyphenyl]-2-naphthalenecarboxylic acid capable of being formulated into the pharmaceutical composition of the invention includes a salt, ester, amide, and the like. The selection of the suitable derivatives may be made by the person skilled in the art based on performance requirement.

The pharmaceutical composition of the invention may be formulated into suitable dose form, such as solutions, suspensions, tablets, dispersible tablets, pills, capsules, powders, sustained release formulations or elixirs, for oral administration or in sterile solutions or suspensions for parenteral administration, by techniques and procedures well known to those skilled in the art.

The pharmaceutical composition of the invention may comprise the active ingredient with a pharmaceutically acceptable carrier. Preferably, the pharmaceutically acceptable carrier is non-toxic and may be selected according to the techniques and procedures conventionally in the art. The concentration of active ingredient in the pharmaceutical composition of the invention will depend on physical factors of the subject in need such treatment, such as the age, body weight, health condition, disease severity, administration route, and other factors, and a suitable treatment regimen and administration amount will be determined by an experienced physicians.

### Method of Treating Cancer Comprising Administration of the Pharmaceutical Composition to a Subject in Need of Such Treatment

The method of treating a cancer provided in the invention comprises administration of 6-[3-(1-adamantyl)-4-methoxyphenyl]-2-naphthalenecarboxylic acid to a subject in need of such treatment.

The method of treating a cancer comprises administration of the pharmaceutical composition of the invention to a subject in a therapeutically effective amount.

The therapeutically effective amounts may be varied based on factors such as the age, sex, body weight, administration route, disease severity of the subject, and other factors. The amount of a given active ingredient will vary depending upon various factors but can nevertheless be routinely determined by one skilled in the art.

The subject in need such treatment of the invention are all members of the animal kingdom including mammals. The mammal in need such treatment of the invention includes human, and suitably the human in need such treatment of the invention is under and/or has received the treatment with Gefitinib or Erlotinib. Preferably, the subject in need of such treatment of the invention has developed a resistance to Gefitinib or Erlotinib.

The method of treating a cancer of the invention further comprises administering to the subject another anti-tumor agent simultaneously, separately or sequentially.

By “a therapeutically effective amount,” means an amount of the active ingredient to induce remission, reduces tumor burden, and/or prevents tumor spread or growth of tumors compared to the response obtained without administration of the active ingredient. The term “treating” or “treatment” is well understood in the art, and means to obtain advantageous or desired results. Advantageous or desired results can include, but are not limited to, alleviation or amelioration of one or more symptoms or conditions, diminishment of tumors, stabilized or not worsening state of tumors, preventing metastasis of tumors, delay or slowing of tumors progression, amelioration or palliation of tumors stage, diminishment of the reoccurrence of tumors, and partial or total remission tumors including prolonging survival as compared to expected survival if not receiving treatment.

### Use of 6-[3-(1-adamantyl)-4-methoxyphenyl]-2-naphthalene-carboxylic acid (or nsl-8) for Manufacturing an Agent for Treating Cancer

The invention further provides a use of 6-[3-(1-adamantyl)-4-methoxyphenyl]-2-naphthalenecarboxylic acid for manufacturing an agent for treating a cancer.

The agent of the invention may be formulated into a dose form suitable for an administration route selected by a person skilled in the art.

The agent of the invention is manufactured for treating a cancer associated with abnormal level of EGFR, and suitably an elevated level of EGFR.

The cancer associated with abnormal level of EGFR is selected from esophageal cancer, gastrointestinal stromal tumor, lung cancer, breast cancer, head and neck cancer, pancreatic cancer, colorectal cancer or glioma cancer.

Further, a combination of the agent of the invention and another anti-tumor agent is provided for treating a cancer. The another anti-tumor agent may be any agent well known in the art suitable for treating a cancer, and preferably Gefitinib or Erlotinib.

The scope of the invention should be well understood with reference to the following EXAMPLES, which are not intended to limit the inventions to the specific embodiments. The specific embodiments disclosed herein are only offered by way of example, and the invention should be limited by the appended claims, along with the full scope of equivalents.

### EXAMPLES

### Preliminary Search Using ADDock:

There were two drug databases being searched by ADDock⁹. The hydrogen atoms for each drug were added. All the crystal waters or cofactor molecules identified inside the docking box of 2ity¹⁰, the structure of wild type EGFR kinase domain determined by X-ray crystallography, were removed before docking. The docking by ADDock for each drug molecule into the active site of 2ity was consisted of the following steps: (i) selecting a terminal atom as an anchor, (ii) counting the topology for the entire molecule based on the anchor selected, (iii) defining the docking parameters and docking box based on the topology coordinates computed for the ligand, and (iv) evolving the parameters using a genetic algorithm and then scoring the interaction between the docked ligand and protein target. ADDock used a piecewise linear potential function to score the interaction between a docked ligand and protein target and both distance and geometry involving in the hydrogen bonding interaction were accounted. The electrostatic interaction energy was computed from the formal charge assigned for each atom and scaled by a distance dependent dielectric constant computed.

Further, ADDock chose anchors by changing all the terminal atoms identified on a ligand systematically. In other words, a series of conformations were automatically generated at the beginning based on all the preliminary anchors chosen and then the best anchor was determined as the minimum docked energy computed among all the preliminary anchors (conformations) generated. The ten docked and top ranked drug molecules by ADDock were selected and tested experimentally for their binding activity with the EGFR kinase domain. Moreover, the ten docked and top ranked drugs into the wild type EGFR kinase domain 2ity were also docked by ADDock into the same kinase domain 4i22¹¹ that carrying double mutations (L858R+T790M) for assessing if there is any difference in binding between them with the wild type and mutant EGFR domains.

### Enzymatic Assay for the Screened Drugs:

Except Tarceva® or Erlotinib which was purchased from LC Laboratories, all the screened compounds were purchased from Sigma-Aldrich. A recombinant wild type human EGFR kinase domain expressed in Sf9 insect cells as a GST-fusion protein was used in the enzymatic assay. The recombinant protein was purified by a GSH-affinity column and the purity of the protein was examined by SDS-PAGE/Coomassie staining and the identity was checked by mass spectroscopy. A radiometric protein kinase assay was used for measuring the kinase activity of the EGFR protein kinase. All the assays were performed in 96-well FlashPlates™ from PerkinElmer (Boston, Mass., USA) in a 50 □L, reaction volume. The 50 □L, assay cocktail contained 70 mM HEPES-NaOH pH 7.5, 3 mM MgCl₂, 3 mM MnCl₂, 3 M Na-orthovanadate, 1.2 mM DTT, 50 μg/ml PEG20000, ATP (corresponding to the apparent ATP-Kₘ determined for the wild type EGFR kinase), [γ-³³P]-ATP (approx. 5×10⁵ cpm per well), 20 ng EGFR kinase, and substrates (variable amounts). The reaction cocktails were incubated at 30° C. for 60 minutes. The reaction was stopped with 50 □L, of 2% (v/v) H₃PO₄ and plates were aspirated and washed two times with 200 □L, 0.9% (w/v) NaCl. Incorporation of ³³Pᵢ was determined with a microplate scintillation counter. All the assays were performed with a BeckmanCoulter/SAGIAN™ Core System.

The median value of the counts in column 1 (n=8) of each assay plate was defined as “low control”. This value reflects unspecific binding of radioactivity to the plate in the absence of a protein kinase but in the presence of the substrate. The median value of the counts in column 7 of each assay plate (n=8) was taken as the “high control”, i.e. full activity in the absence of any inhibitor. The difference between high and low control was taken as 100% activity. The low control value from a particular plate was subtracted from the high control value as well as from all 80 “compound values” of the corresponding plate. The residual activity (in %) for each well of a particular plate was calculated by using the following formula:

Res. Activity (%)=100×[(cpm of compound−low control)/(high control−low control)]

The residual activities for each concentration and the drug IC₅₀ values were calculated using GraphPad Prism software. The fitting model for the IC₅₀ determinations was “Sigmoidal response (variable slope)” with parameters “top” fixed at 100% and “bottom” at 0%. The fitting method used was a least-squares fit.

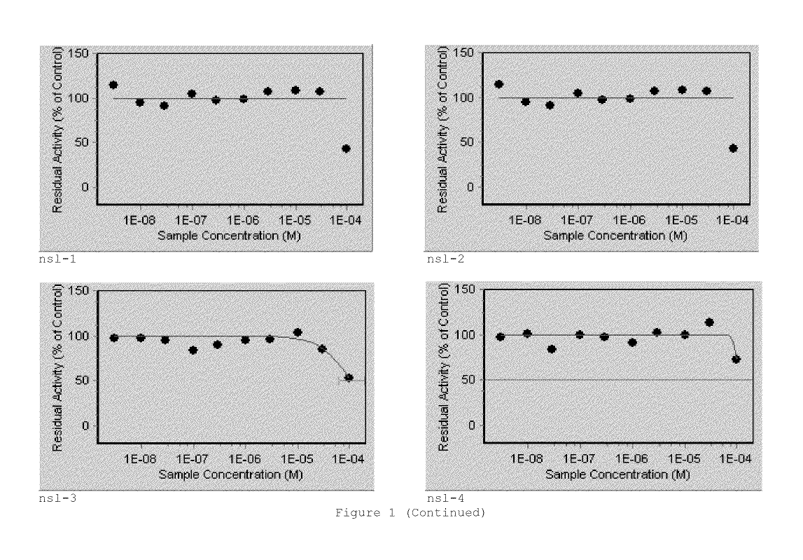

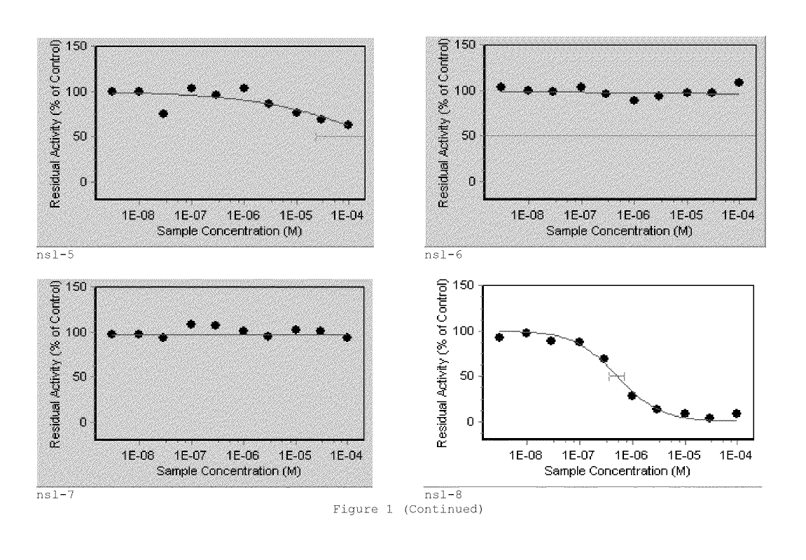

The docked energies given by ADDock for the 10 selected drugs nsl-1 to nsl-10 into the active site of wild type EGFR kinase domain 2ity were −76.2, −109, −94.1, −89.8, −79.5, −82.1, −90.6, −99.7, −94.3, and −92.4 kcal/mol, respectively. The IC₅₀ determined by the enzymatic assay for the 10 selected drugs (nsl-1 to nsl-10) by ADDock were listed in Table 1. There were 10 concentrations used for determining the IC₅₀ for each drug and the results were presented in FIG. 1 for each drug tested. Apparently, with IC₅₀ measured as 0.515 μM (FIG. 1), only drug nsl-8 was considered to be active in binding with the wild type EGFR kinase domain. Note that the docked energy given by ADDock for drug nsl-8 was ranked as the second best one among all the 10 drugs studied.

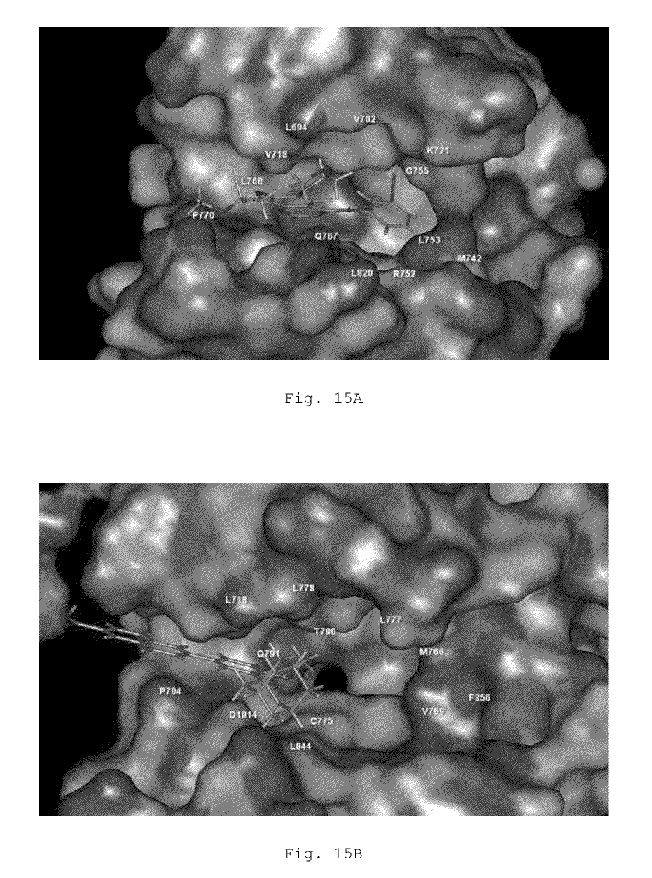

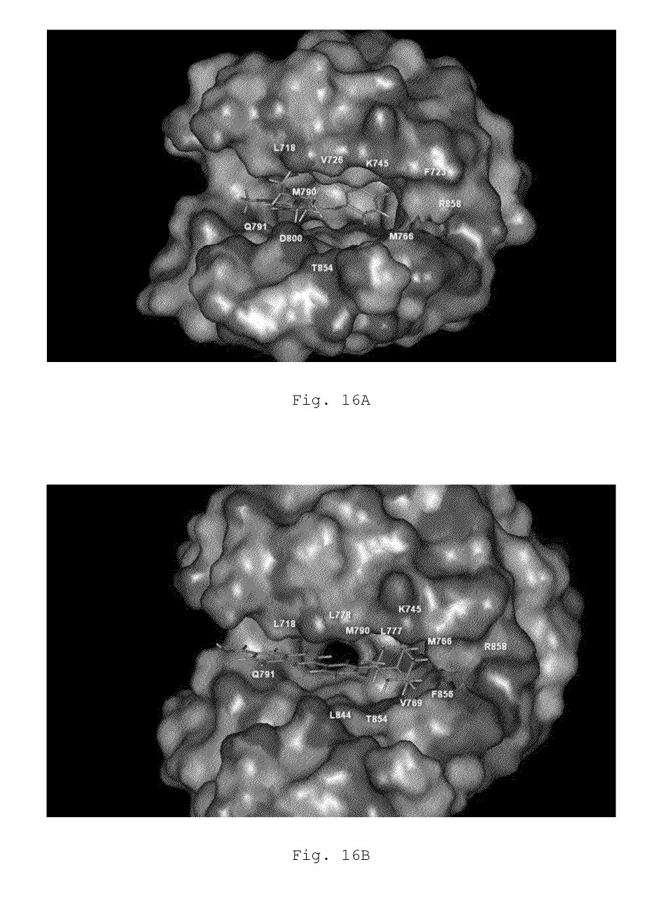

We have also docked Tarceva® or Erlotinib into the active sites of both wild type 2ity and mutant EGFR kinase domain 4i22 carrying two point mutations L858R+T790M using ADDock. As shown in FIGS. 15a and 15b, there is a significant difference in binding with the active site residues of wild type EGFR kinase domain between nsl-8 and Tarceva® or Erlotinib. While the ethynylphenyl group of Tarceva® or Erlotinib was deeply docked into the catalytic pocket, the quinazolin and the two 2-methoxyethoxy groups of the drug were docked respectively into the neck and rim regions of the pocket (FIG. 15a). Note that the root-mean-square (rms) deviation computed between the docked conformation by ADDock and the X-ray determined structure was 0.08 Å. However, as shown in FIG. 15b, while the methoxy-phenyl associated naphthalene group of nsl-8 was docked around the outer neck region, the adamantyl group of nsl-8 was docked into a upper hydrophobic cleft of the catalytic pocket. The docked energy by ADDock for nsl-8 and Tarceva® or Erlotinib into the active site of wild type enzyme 2ity obtained were −99.7 and −119 kcal/mol, respectively. This demonstrated that the binding mode of nsl-8 with wild type EGFR kinase domain was quite different from that of Tarceva® or Erlotinib with the wild type enzyme. This is also true for docking both nsl-8 and Tarceva® or Erlotinib into the active site of mutant EGFR kinase domain 4i22. The docked energy by ADDock for nsl-8 and Tarceva® or Erlotinib into the active site of mutant enzyme 4i22 obtained were −115 and −118 kcal/mol, respectively. Apparently, there was no significant difference in docking results exhibited by Tarceva® or Erlotinib into the mutant (FIG. 16a) or wild type enzymes (FIG. 15a). However, there was a clear-cut difference found between docking nsl-8 into the mutant (FIG. 16b) and wild type enzymes (FIG. 15b), respectively. The adamantyl group of nsl-8 appeared to be docked into a different hydrophobic cleft of mutant (FIG. 16b) to that of wild type enzyme (FIG. 15b). This indicated that the binding mode of nsl-8 with mutant EGFR domain was also quite different from that of Tarceva® or Erlotinib with mutant enzyme. This would imply that nsl-8 may be useful for treating patients who have developed drug resistance after being treated with Tarceva® or Erlotinib.

TABLE 1
Screening of a drug lead through measuring IC₅₀
with an expressed wild type EGFR kinase domain.

| | | | | EGF-R wt |
|---|---|---|---|---|
| # | Compound ID | Plate ID | Well ID | IC₅₀ (M) |
| 1 | nsl-1 | 7482 TAIWA1 | A2 | >1E−04 |
| 2 | nsl-2 | 7482 TAIWA1 | B2 | 9.00E−05 |
| 3 | nsl-3 | 7482 TAIWA1 | C2 | >1E−04 |
| 4 | nsl-4 | 7482 TAIWA1 | D2 | >1E−04 |
| 5 | nsl-5 | 7482 TAIWA1 | E2 | >1E−04 |
| 6 | nsl-6 | 7482 TAIWA1 | F2 | >1E−04 |
| 7 | nsl-7 | 7482 TAIWA1 | G2 | >1E−04 |
| 8 | nsl-8 | 7482 TAIWA1 | H2 | 5.15E−07 |
| 9 | nsl-9 | 7482 TAIWA2 | A2 | >1E−04 |
| 10 | nsl-10 | 7482 TAIWA2 | B2 | >1E−04 |
| 11 | DMSO | 7482 TAIWA2 | C2 | >1E−04 |
| 12 | DMSO | 7482 TAIWA2 | D2 | >1E−04 |
| 13 | DMSO | 7482 TAIWA2 | E2 | >1E−04 |
| 14 | DMSO | 7482 TAIWA2 | F2 | >1E−04 |
| 15 | DMSO | 7482 TAIWA2 | G2 | >1E−04 |
| 16 | DMSO | 7482 TAIWA2 | H2 | >1E−04 |

### 2D Cell Proliferation Assay for the Drug Lead Searched:

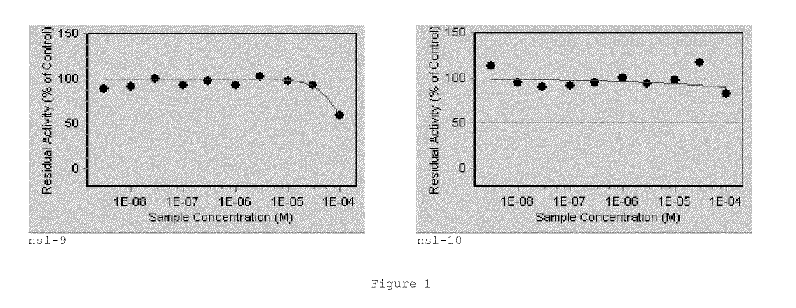

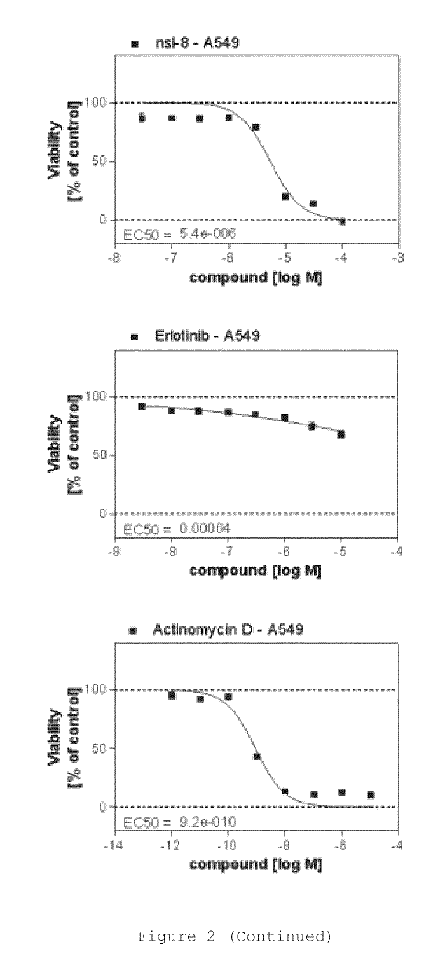

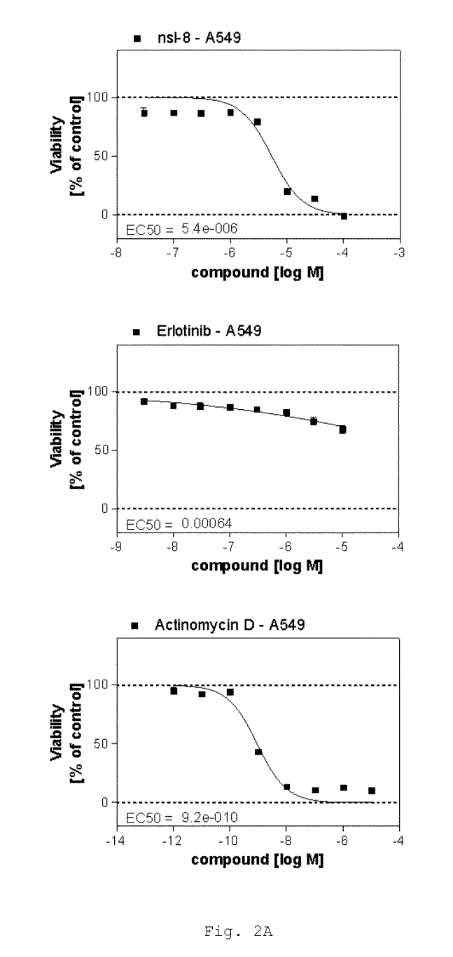

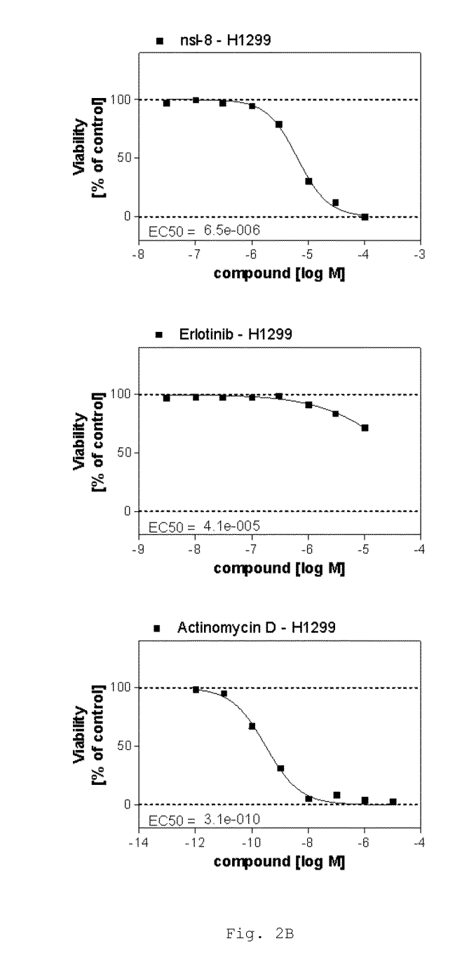

Nsl-8 was subjected to 2D cell proliferation assays using four NSCLC cell lines (A549 (human adenocarcinomic alveolar basal epithelial cells with k-ras gene mutation), H1299 (human non-small cell lung carcinoma cell line with p53 mutation), H460 (human non-small cell lung carcinoma cell line with with k-ras gene mutation), and HCC827 (human NSCLC cells with EGFR mutation of deletion from E746 to A750)) and the results were presented in Table 2. There were eight concentrations used for each drug for determining its IC₅₀. The corresponding plots of activity against concentration were presented in FIG. 2. Note that Tarceva® or Erlotinib, a FDA 2004 approved NSCLC drug was included in the assays for comparison. Tarceva® or Erlotinib showed partial inhibition of the growth of HCC827 down to the lowest concentration, indicating that the compound has an IC₅₀ below 3.0 nM. Except this cell line, all the other IC₅₀ measured for Tarceva® or Erlotinib were greater than 10 μM (Table 2) which were in accord with those published by others previously^{12,13}. Actinomycin D was a chemotherapeutic drug simply used as a quality control for all the experiments.

In the 2D cell proliferation assay, cells were cultured in DMEM containing 10% FCS and Penicillin/Streptomycin. For the assays, cells were seeded in 150 μL medium on a 96-well cell culture plate and incubated at 37° C. overnight before the compounds were added. Compounds were prepared as predilution which was 16 fold concentrated to the final assay concentration. A day after cell seeding, 10 μL of prediluted compounds was added to the cells (1:16 dilution). Treatment of cells with 0.1% DMSO or 1% DMSO (for the two top concentrations of nsl-8) and 1.0E-05 M Staurosporine was served as high control (100% viability) and low control (0% viability), respectively. Measurement of the impact of compounds on cell viability was performed as follows: 5000 cells (A549 and HCC827) or 2500 cells (H1299 and H460) were seeded in the inner wells of 96-well-plates in 150 μL complete medium. Next day, compounds were added to the medium to reach the final concentration and then incubated at 37° C. in 5% or 10% CO₂ depending on the medium for 72 h. Subsequently 10 μL Alamar Blue reagent was added and fluorescence at 590 nm was measured after 5 h incubation at 37° C. in 5% CO₂ using a fluorometer.

Raw data were converted into percent cell viability relative to high control (solvent 0.1% DMSO or 1% DMSO for the two top concentrations of drug nsl-8) and low control (1.0E-05 M Staurosporine), which were set to 100% and 0%, respectively. IC₅₀ calculation was performed using GraphPad Prism software with a variable slope sigmoidal response fitting model using 0% cell growth as bottom constraint and 100% cell growth as top constraint. As shown in FIG. 2 and Table 2 for the four NSCLC cell lines tested, the IC₅₀ determined for nsl-8 were varied from 2 to 11 μM, indicating that the drug was effective in retarding the growth of each of these four NSCLC cell lines.

TABLE 2
2D cell-based assays for measuring IC₅₀ for
nsl-8 using four different NSCLC cell lines.

| | | nsl-8 | Erlotinib | Actinomycin D |
|---|---|---|---|---|
| Plate ID | Cell type | IC₅₀ [M] | IC₅₀ [M] | IC₅₀ [M] |
| 1784 | A549 | 5.4E−06 | >1.0E−05 | 9.2E−10 |
| 1785 | H1299 | 6.5E−06 | >1.0E−05 | 3.1E−10 |
| 1786 | H460 | 2.0E−06 | >1.0E−05 | 4.2E−10 |
| 1787 | HCC827 | 1.1E−05 | partial inhibition | 1.2E−08 |

### 3D Cell Proliferation Assay for the Drug Lead Searched:

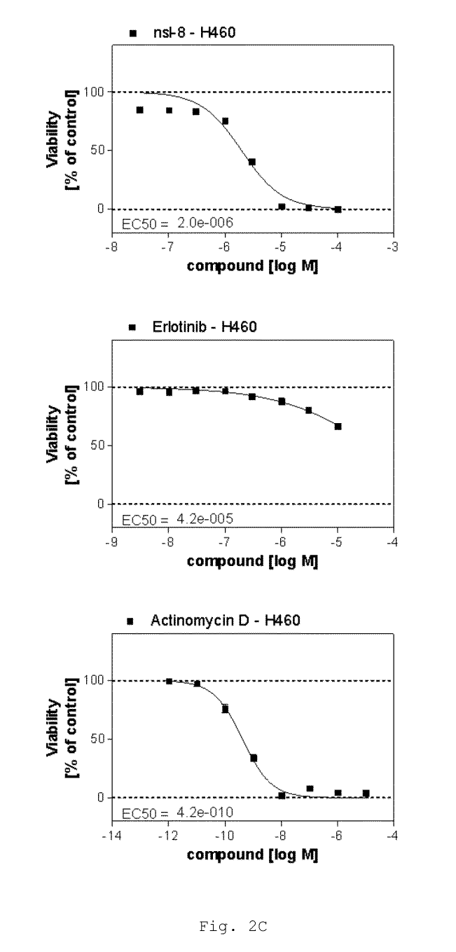

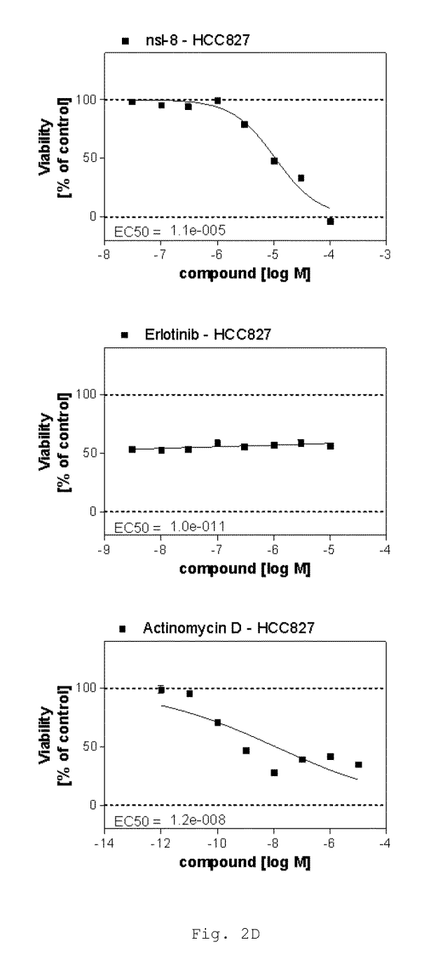

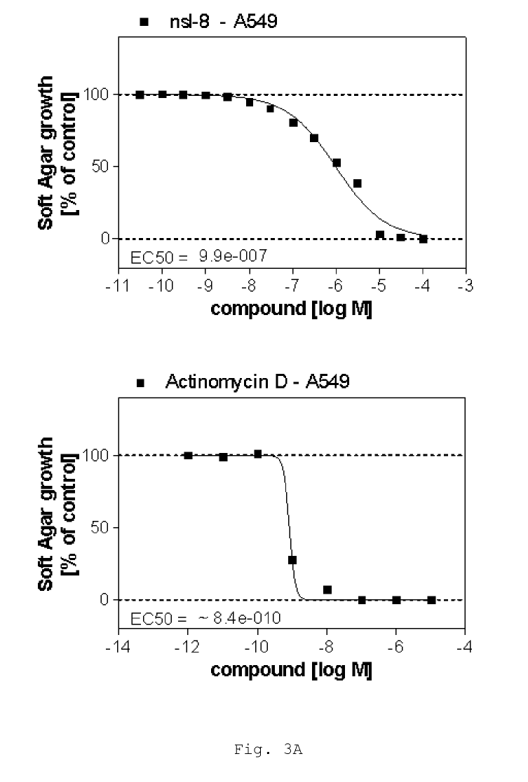

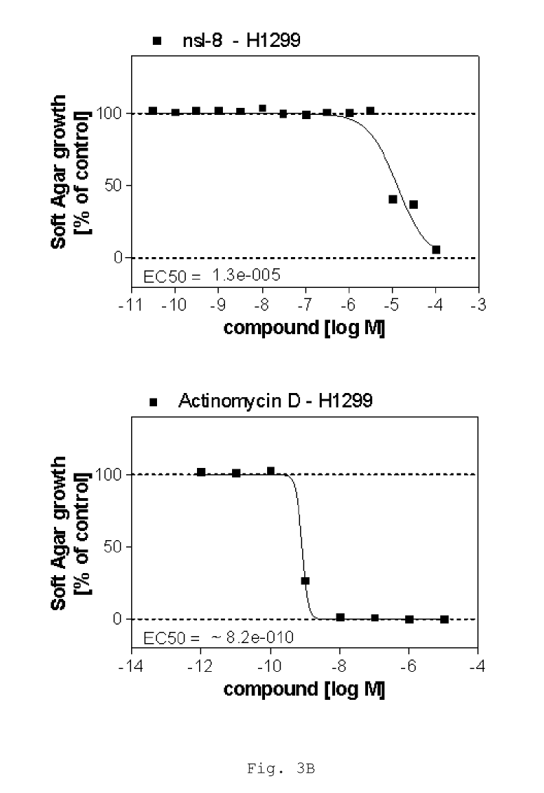

Drug nsl-8 was further subjected to a 3D cell proliferation assay for assessing its potential in inhibiting the growth of NSCLC tumors. The same four NSCLC cell lines used in 2D proliferation assays were grown in soft agar and the drug was added after the agar has solidified. The drug added cells were then incubated several days until colonies have formed in the solvent control. Subsequently, Alamar Blue which was a cell viability reagent was added and the corresponding fluorescence released was measured as an indirect quantification of colony growth in soft agar. There were fourteen concentrations prepared for determining the IC₅₀ for each drug and the results were presented in FIG. 3.

For each cell line, some 96 well culture plates were prepared. 100 μL of the soft agar bottom layer (0.6% final concentration in complete medium) was poured and left to solidify. 50 μL of the soft agar top layer (0.4% final concentration) containing the corresponding cells and cell number were then added on top, solidified and such 96 well plates were incubated overnight at 37° C. in 10% CO₂. Next day, drugs were added at indicated final concentrations into the inner wells of the plate. Subsequently, the assays were incubated in cell culture incubators for the indicated period of time. Finally, the assays were developed using Alamar Blue and upon 3-5 h of incubation at 37 ° C. fluorescence intensity was determined (excitation: 560 nm, emission: 590 nm). As a low control, cells were treated with 1.0E-05M Staurosporine (6 fold values). As a high control, cells were treated with 0.1% DMSO (solvent control, 6 fold values). The raw data were treated using the same procedure described for the 2D cell proliferation assay. The IC₅₀ determined for nsl-8 in each of the four NSCLC cell lines were summarized in Table 3. The IC₅₀ determined for nsl-8 in these four cell lines varied from 0.99 to 13 μM, indicating again that the drug was effective in inhibiting the growth of these four NSCLC cell lines.

TABLE 3
3D cell-based assays for measuring IC₅₀ for
nsl-8 using four different NSCLC cell lines.

| | | nsl-8 | Actinomycin D |
|---|---|---|---|
| Plate ID | Cell type | IC₅₀ [M] | IC₅₀ [M] |
| 1366 | A549 | 9.9E−07 | 8.4E−10 |
| 1367 | H1299 | 1.3E−05 | 8.2E−10 |
| 1368 | H460 | 4.9E−06 | 6.7E−10 |
| 1369 | HCC827 | 7.2E−06 | 2.4E−10 |

### Mouse Xenograft Models for the Drug Lead Searched:

The antitumor efficacy of the drug lead nsl-8 searched was studied using the subcutaneous xenografted A549 and HCC827 lung cancer tumor models in vivo. As detailed in Table 4, the study consisted of 8 experimental groups each containing either 8 female BALB/c nude mice (A549: groups 1a and 1b) or 10 female SCID mice (A549: groups 2a and 2b, HCC827: groups 3a and 3b) for treating with nsl-8, plus 5 female SCID mice (A549: group 2c, HCC827: group 3c) for treating with Tarceva® or Erlotinib. All experiments were performed using either 5-6 weeks old female BALB/c nude mice (approximate weight: 16-20 g) (A549) or female SCID mice (approximate weight: 16-20 g) (A549 and HCC827). Mice were maintained in individual ventilated cages (IVC, max. 4 mice/cage) at constant temperature and humidity. Animal weights were determined three times (Monday, Wednesday and Friday) for groups 1a and 1b or two times (Monday and Thursday) for groups 2a-2c and 3a-3c animals. Animal behaviour was monitored daily.

On day 0, either 2×10⁶ A549 or 2×10⁶ HCC827 tumor cells in 100 μL PBS were implanted into the subcutaneous space of the left flank of all corresponding BALB/c nude and SCID mice, respectively. In the following, tumor sizes were determined by caliper measurement. Tumor sizes were calculated according to the formula W²×Lg/2 (Lg=length and W=the perpendicular width of the tumor, L>W). On day 48 or 30, after mean tumor volumes had reached approx. 100 mm³, the corresponding tumor-bearing animals were randomized into 2 groups of 8 (A549 model: groups 1a and 1b) or 10 animals (A549 model: groups 2a and 2b) according to tumor sizes. On day 24 or 28, after mean tumor volumes had reached approx. 100 mm³, the corresponding tumor-bearing animals were randomized into 2 groups of 10 animals (HCC827 model: groups 3a and 3b) according to tumor sizes. The third groups (2c and 3c) with 5 animals assigned for each were reserved for treating with Tarceva® or Erlotinib. The percentage of tumor growth inhibition (TGI) was calculated using the following formula:

%TGI=[1−(T/C)]×100%

where T and C represent the mean tumor volume of the treatment and the control group, respectively.

On the same day, treatment with drug nsl-8, Tarceva® or Erlotinib, and vehicle control was initiated (see Table 4 for details). As shown in Table 4, except groups 1a and 1b where animals were treated with i.p. route, animals in all the other groups were treated with o.p. route. For preparing the daily nsl-8 dosage solutions for treating groups 1a and 1b, 158.4 mg nsl-8 was weighed and dissolved in 19.5 ml DMSO resulting in a final concentration of 8.123 mg drug per ml DMSO. The solution was aliquoted into several 15 ml sterile centrifuge tubes in 650 μl quantity per tube and then stored frozen at −80° C. until use. To make a daily dosage solution for injecting groups 1a or 1b animals, an aliquot was taken from the −80° C. refrigerator and thawed on ice and then mixed well with 5.85 ml of 0.5% w/v methyl cellulose (Sigma; M0512-110 g; Lot #079K0054V) in PBS pH 7.4 (PAA; H21-002; Lot-#H00212-1920), i.e. a 10% nsl-8 DMSO stock solution in methyl cellulose suspension was prepared. As shown in Table 4, animals of vehicle control groups 1a, 2a, and 3a received 49.24 ml/kg vehicle through i.p. route once daily on days 48-66 and 74.36 ml/kg vehicle through o.p. route once daily on days 1-14, respectively. Animals of group 1b received 40 mg/kg nsl-8 dosage solution once daily through i.p. route on days 48-52 and 62-66, while those of groups 2b and 3b received 70 mg/kg nsl-8 dosage solution once daily through o.p. route on days 1-14 (group 2b) and days 1-14 (group 3b), respectively (Table 4). Moreover, animals of groups 2c and 3c received 20 mg/kg Tarceva® or Erlotinib through o.p. route once daily on days 1-14 (Table 4).

TABLE 4
The study design for testing antitumor efficacy of nsl-8 using
subcutaneously implanted A549 and HCC827 xenograft models.

| | | Tumor model & | | Application | | Animal |
|---|---|---|---|---|---|---|
| Group name | | (mouse strain) | Treatment | Route | Scheme | Number |
| 1a | vehicle | A549 | 49.24 | i.p. | 1x | 8 |
| | control | (BALB/c nude) | ml/kg | | daily | |
| | | | | | on days | |
| | | | | | 48-52 | |
| | | | | | and 62- | |
| | | | | | 66 | |
| 1b | nsl-8 | A549 | 40 mg/kg | i.p. | 1x | 8 |
| | | (BALB/c nude) | | | daily | |
| | | | | | on days | |
| | | | | | 48-52 | |
| | | | | | and 62- | |
| | | | | | 66 | |
| 2a | vehicle | A549 | 74.36 | o.p. | 1x | 1C |
| | control | (SCID) | ml/kg | | daily | |
| | | | | | on days | |
| | | | | | 1-14 | |
| 2b | nsl-8 | A549 | 70 mg/kg | o.p. | 1x | 1C |
| | | (SCID) | | | daily | |
| | | | | | on days | |
| | | | | | 1-14 | |
| 2c | Tarceva ® | A549 | 20 mg/kg | o.p. | 1x | 5 |
| | | (SCID) | | | daily | |
| | | | | | on days | |
| | | | | | 1-14 | |
| 3a | vehicle | HCC827 | 74.36 | o.p. | 1x | 1C |
| | control | (SCID) | ml/kg | | daily | |
| | | | | | on days | |
| | | | | | 1-14 | |
| 3b | nsl-8 | HCC827 | 70 mg/kg | o.p. | 1x | 1C |
| | | SCID) | | | daily | |
| | | | | | on days | |
| | | | | | 1-14 | |
| 3c | Tarceva ® | HCC827 | 20 mg/kg | o.p. | 1x | 5 |
| | | (SCID) | | | daily | |
| | | | | | on days | |
| | | | | | 1-14 | |

Groups 1a and 1b (A549 model) were terminated on day 69, while groups 2a-2c (A549 model) and 3a-3c (HCC827 model) were terminated on day 15. At necropsy, animals were killed by cervical dislocation. Primary tumor weights and volumes were determined. The tumor volume and weight were analysed using descriptive data analysis (Mean, SEM, Median). Statistical analysis of efficacy data was performed using the unpaired t-test. All the data analyses were performed using GraphPad Prism 5 from GraphPad Software, Inc., San Diego, USA.

The mouse serums of groups 2a-2c and 3a-3c animals at the end-point of experiment were also collected for assessing if their corresponding renal and liver are functioning normally after being treated by vehicle, nsl-8 or Tarceva® or Erlotinib. The serum level of blood urea nitrogen (BUN) was measured for assessing the renal function while those of alanine aminotransferase (ALT) and aspartate aminotransferase (AST) were measured using Ektachem DT system (Johnson & Johnson Clinical Diagnostics) for assessing the liver function. The EDTA treated anticoagulated blood samples were also collected for making a complete peripheral blood count by using a Hematology Analyzer (Sysmex, XT-1800i).

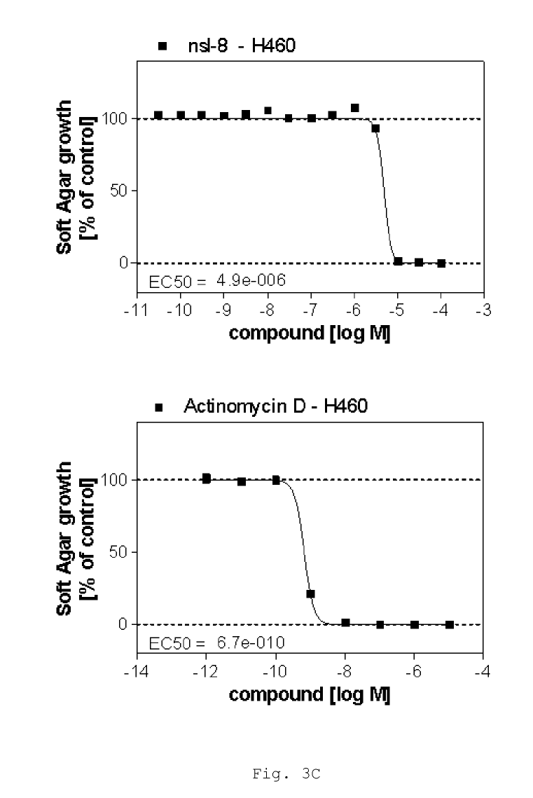

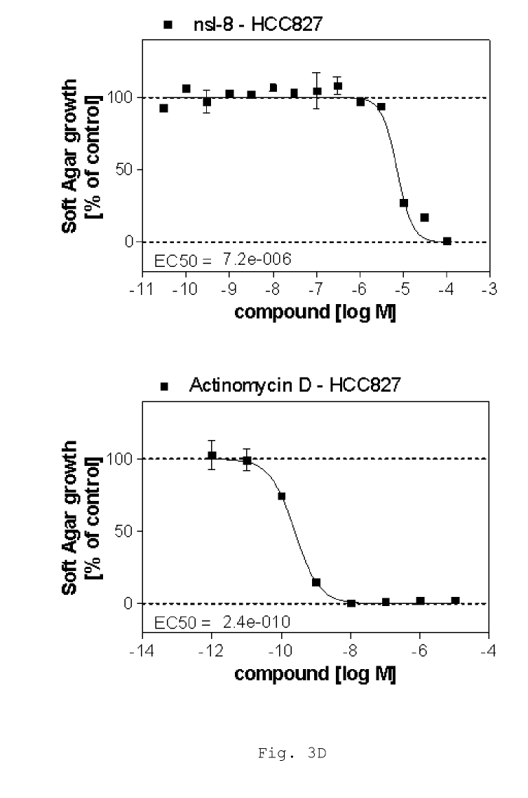

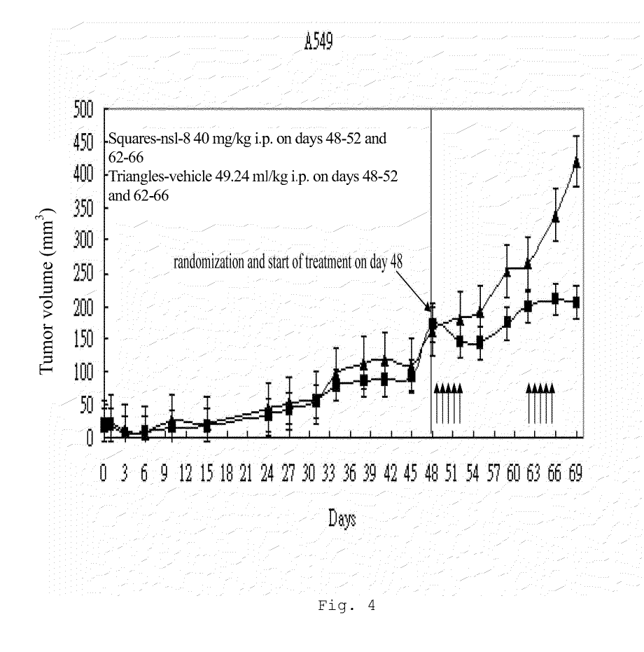

The treatment for groups 1a and 1b (A549 model) animals with drug nsl-8 was started on day 48 and terminated on day 69. The animals were not consecutively treated with nsl-8 during the entire treatment period of 48-68 days. Instead, there was an interruption between treatment days 48-52 and 62-66 (FIG. 4) due to an apparent weight loss observed in the treated animals, an ethical reason which caused the treatment to be suspended. Indeed, as shown in FIG. 5, a 16% weight loss in animals after being treated with nsl-8 from days 48-52 was observed. However, the animals were able to recover from such a weight loss after several days of cessation of treatment (FIG. 5). After weight loss of treated animals was recovered, a second treatment cycle from days 62-66 was resumed (FIG. 5). This second treatment cycle caused weight loss again (FIG. 5). However, the study groups 1a and 1b were both terminated on day 69 due to the presence of tumor ulceration in some animals. Note that weight loss in animals treated with vehicle was also observed in the final stage of treatment (FIG. 5). We speculated that such a loss might be caused by the presence of 10% DMSO in the prepared vehicle. However, as shown in FIGS. 4 and 6, a significant reduction in tumor size (P=0.0838) or noticeable antitumor efficacy of nsl-8 was observed after the study had been completed on day 69. As shown in FIG. 6, the treatment with 40 mg/kg nsl-8 through i.p. route caused about 50% reduction in tumor size as compared with that of the vehicle group. The antitumor efficacy of nsl-8 could have been more significant if no interruption was imposed due to the ethical reason during the entire treatment period.

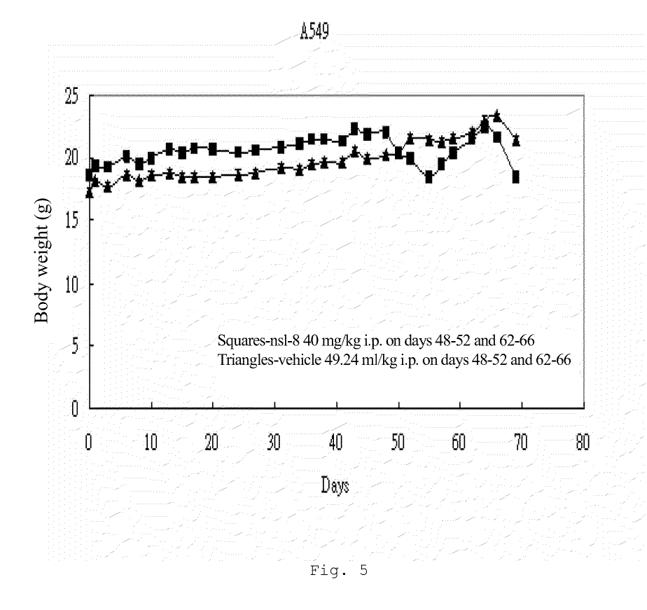

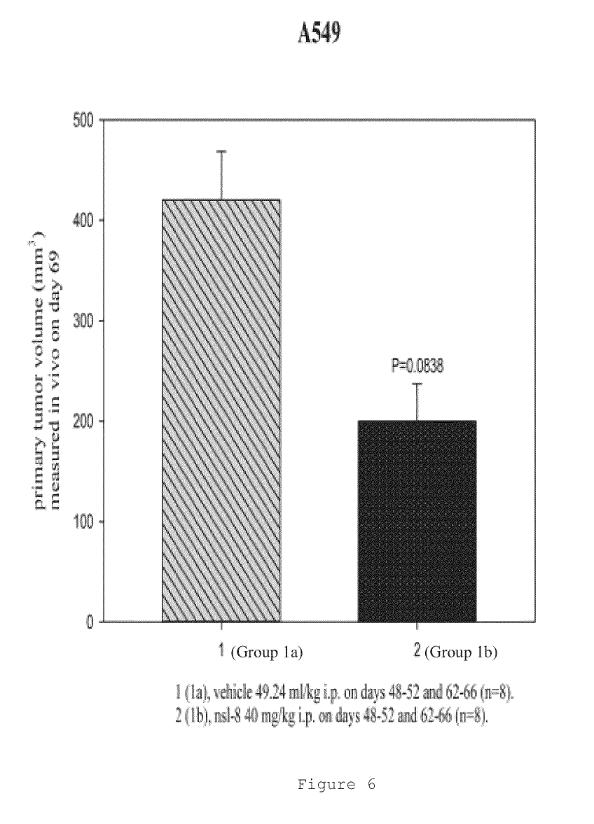

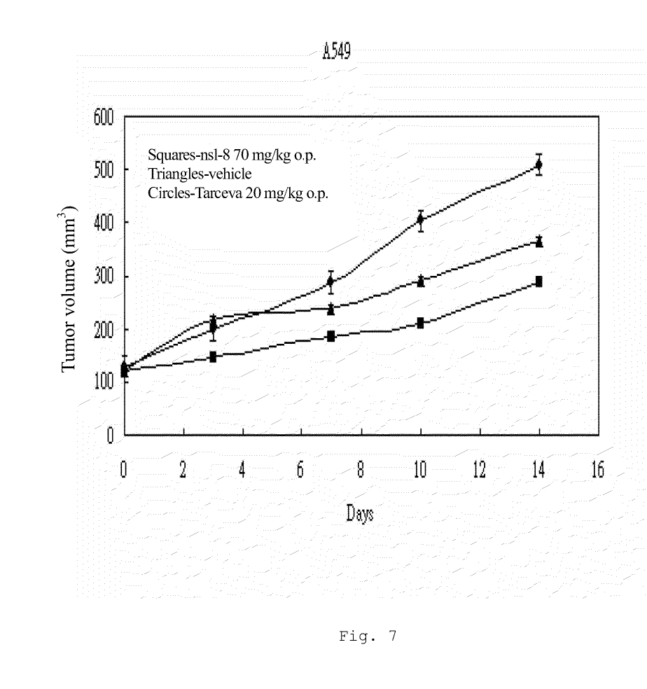

To further study the antitumor efficacy of drug nsl-8 in vivo, we have administered 20 SCID mice of groups 2b and 3b with a dosage of 70 mg/kg through o.p. route, respectively. We also employed Tarceva® or Erlotinib as a control in this study by fitting 10 SCID mice of groups 2c and 3c with a dosage of 20 mg/kg through o.p. route, respectively. For this study, 0.161 ml nsl-8 stock solution made in DMSO was mixed with 1.339 ml 0.9% w/v saline so that the volume of a final dosage solution prepared to feed an animal was 1.5 ml and containing 10% DMSO. Therefore, both administration routes and vehicles used in treating groups 2a-2b and 3a-3b animals were quite different from that used in treating groups 1a-1b animals. As shown in FIG. 7, the growth of A549 implanted tumors in group 2b animals fed with 70 mg/kg nsl-8 daily was gradually inhibited with an increasing rate from 7.1 to 19.1% during the 14 experimental days. On the contrary, there was no inhibition in growth of A549 implanted tumors exhibited by group 2c animals fed with 20 mg/kg Tarceva® or Erlotinib (FIG. 7). This was in accord with those presented in Table 2 where the growth of A549 cell line was barely inhibited by Tarceva® or Erlotinib added. However, as shown in Table 2 and FIG. 8, Tarceva® or Erlotinib was very effective in inhibiting the growth of HCC827 cell line or the HCC827 tumor bearing animals fed with a dosage of 20 mg/kg daily. This also served as a quality control of our experiment. As shown in FIG. 8, the growth of HCC827 implanted tumors in group 3b animals fed with 70 mg/kg nsl-8 daily was also gradually inhibited with an increasing rate from 16.6 to 28.7% during the entire experimental period. Therefore, these in vivo experiments (both FIGS. 7 and 8) further confirmed the efficacy of nsl-8 in treating both A549 and HCC827 implanted tumors.

TABLE 5
The tumor inhibition rates of nsl-8 and Tarceva
estimated in the A549 implanted tumors.

| | Tumor inhibition rate (%) | | | | |
|---|---|---|---|---|---|
| Treatment | Day 0 | Day 3 | Day 7 | Day 10 | Day 14 |
| NSL-8 70 mpk | 0 | 7.1 | 12.0 | 17.8 | 19.1 |
| Tarceva 20 mpk | 0 | −16.7 | −19.0 | −22.0 | −29.6 |

TABLE 6
The tumor inhibition rates of nsl-8 and Tarceva
estimated in the HCC827 implanted tumors.

| | Tumor inhibition rate (%) | | | | |
|---|---|---|---|---|---|
| Treatment | Day 0 | Day 3 | Day 7 | Day 10 | Day 14 |
| NSL-8 70 mpk | 0 | 16.6 | 13.8 | 22.4 | 28.7 |
| Tarceva 20 mpk | 0 | 31.0 | 48.7 | 50.5 | 68.0 |

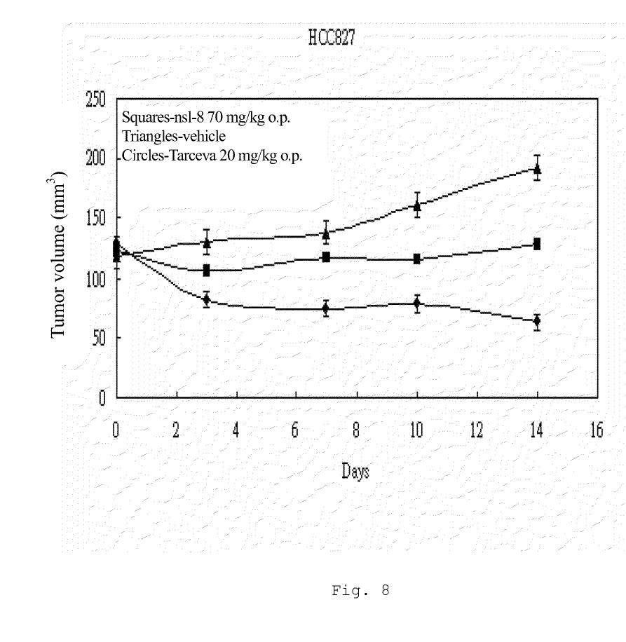

The change in body weight of groups 2a-2c and 3a-3c animals during the entire experimental period was also monitored and presented in FIGS. 9 and 10 and Tables 7 and 8, respectively. There was a minor decreasing in body weight of groups 2b and 3b animals fed with 70 mg/kg nsl-8 daily during the entire experimental period. A decline in 2.0 or 3.8% of body weight of group 2b or 3b animals was observed upon the completion of the experiments (FIGS. 9 and 10 and Tables 7 and 8). This was not in violation with the ethical reason as opposite to that shown in FIG. 5 for group 1b animals where 40 mg/kg nsl-8 was injected daily through i.p. route.

TABLE 7
The effect of nsl-8 and Tarceva on the body
weight of A549 tumor-bearing mice.

| | Body weight change (%) | | | | |
|---|---|---|---|---|---|
| Treatment | Day 0 | Day 3 | Day 7 | Day 10 | Day 14 |
| Vehicle | 0 | 0.3 | 0.7 | 0.1 | 0.3 |
| NSL-8 70 mpk | 0 | −1.9 | −1.3 | −2.6 | −2.0 |
| Tarceva 20 mpk | 0 | 2.2 | 3.4 | 3.5 | 2.7 |

TABLE 8
The effect of nsl-8 and Tarceva on the body
weight of HCC827 tumor-bearing mice.

| | Body weight change (%) | | | | |
|---|---|---|---|---|---|
| Treatment | Day 0 | Day 3 | Day 7 | Day 10 | Day 14 |
| Vehicle | 0 | 0 | 1.3 | −0.1 | −0.5 |
| NSL-8 70 mpk | 0 | −0.2 | 0.2 | −1.9 | −3.8 |
| Tarceva 20 mpk | 0 | 0.2 | 1.3 | 0.6 | −0.2 |

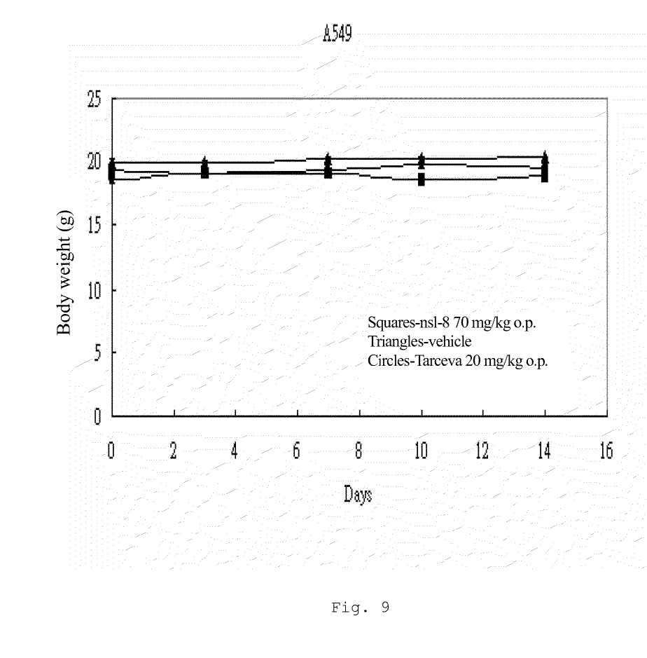

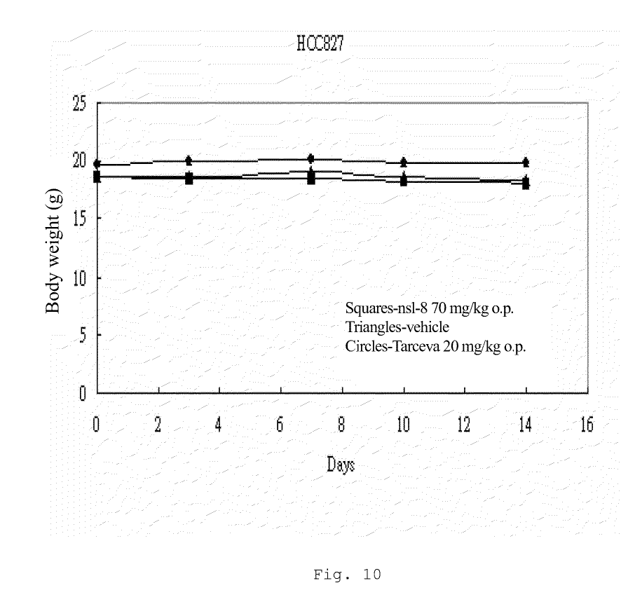

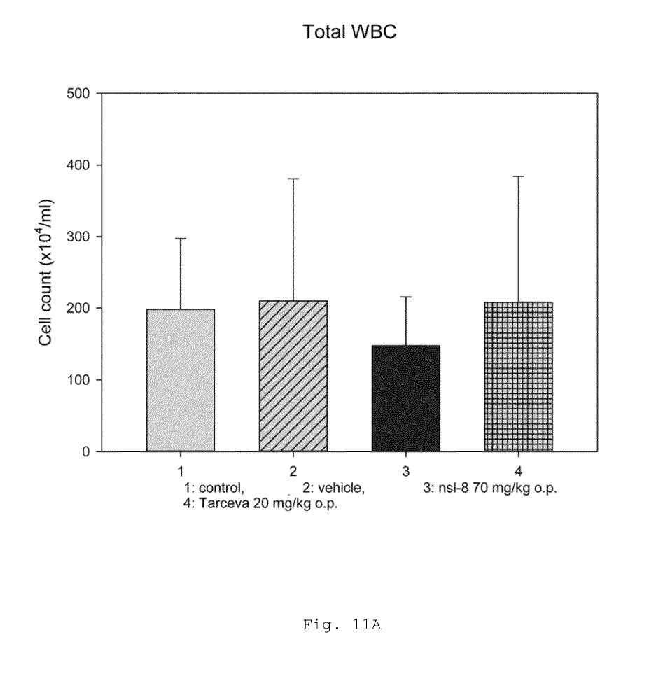

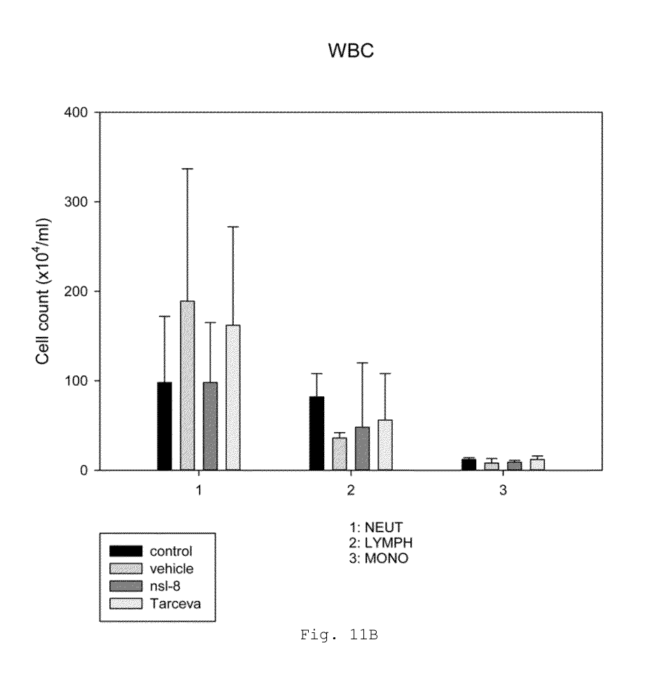

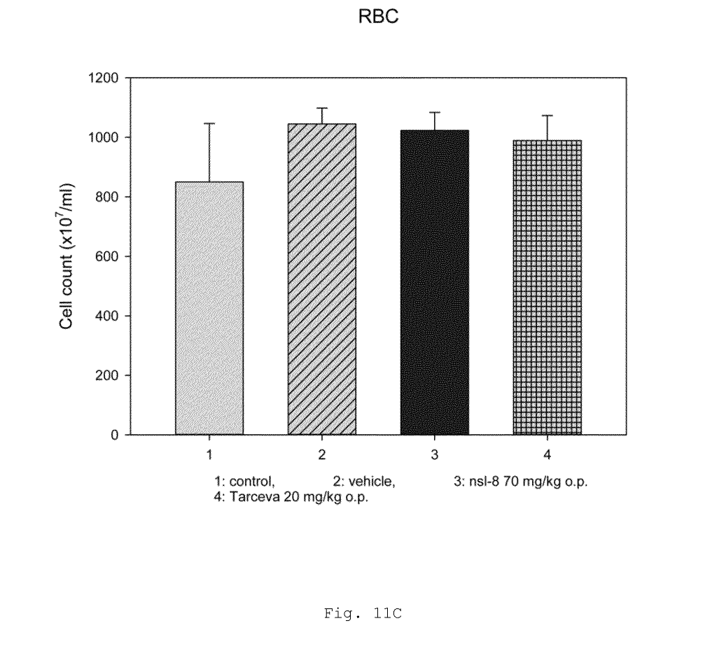

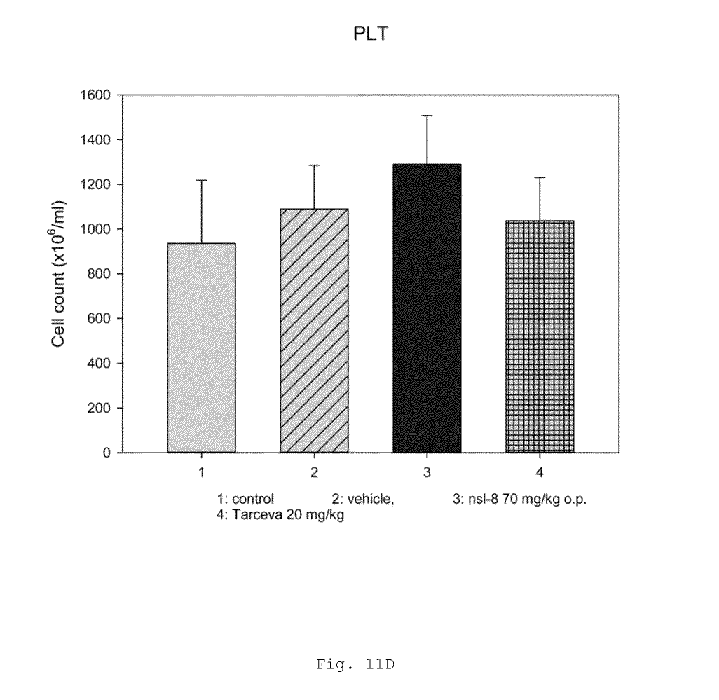

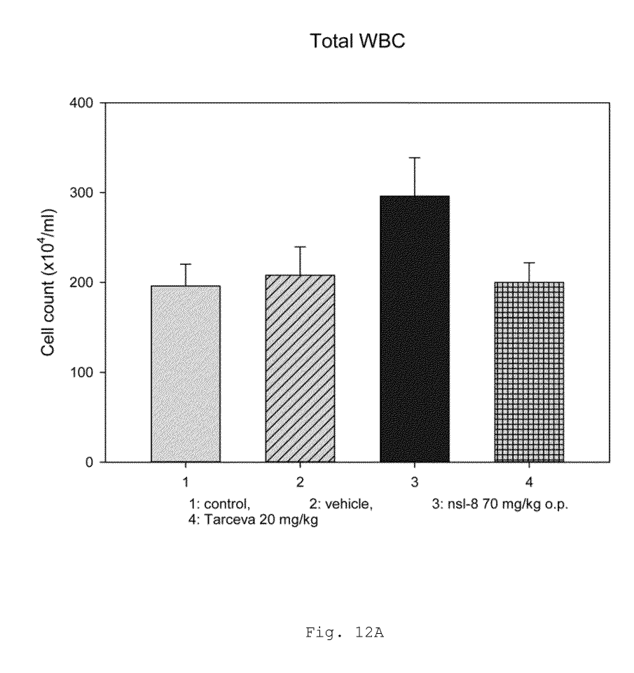

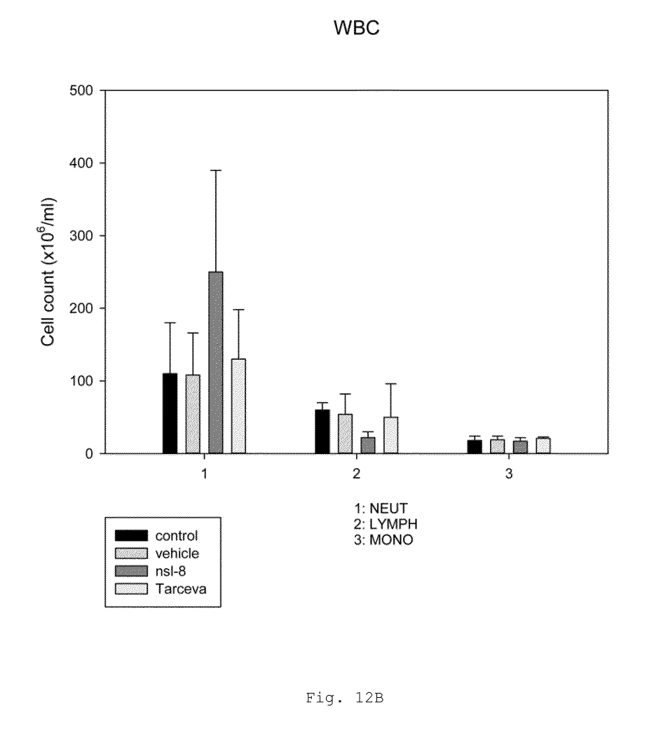

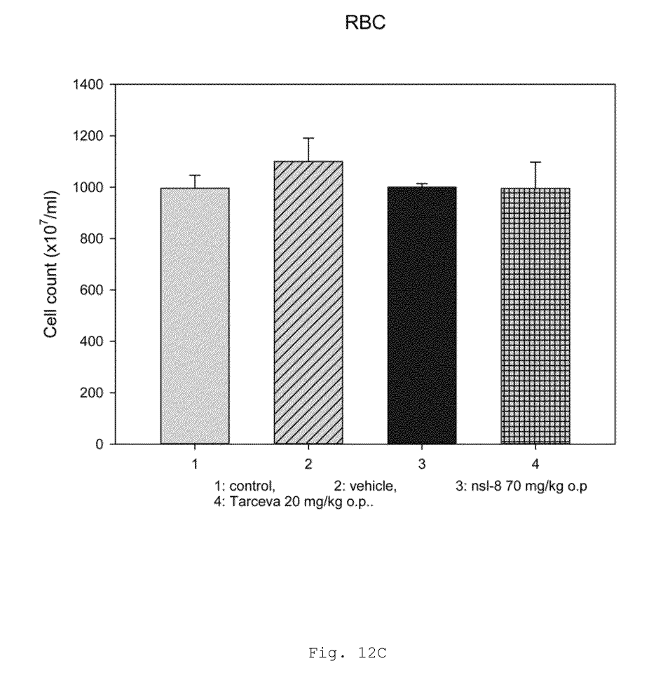

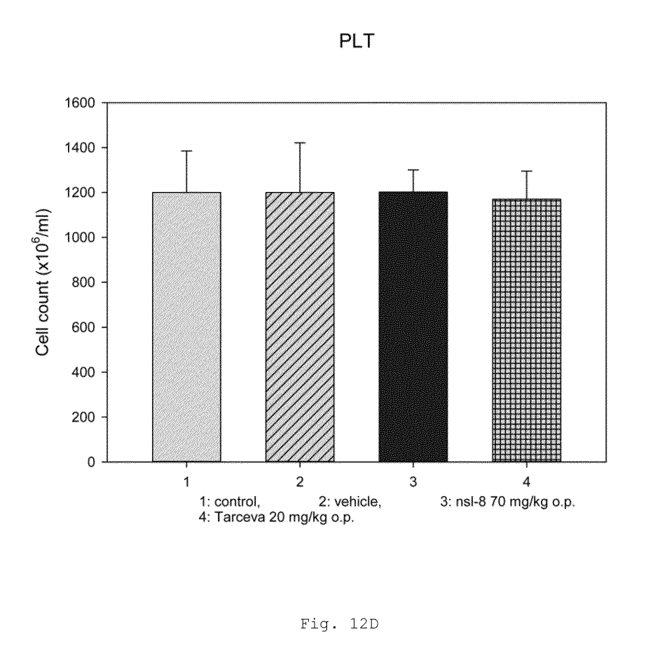

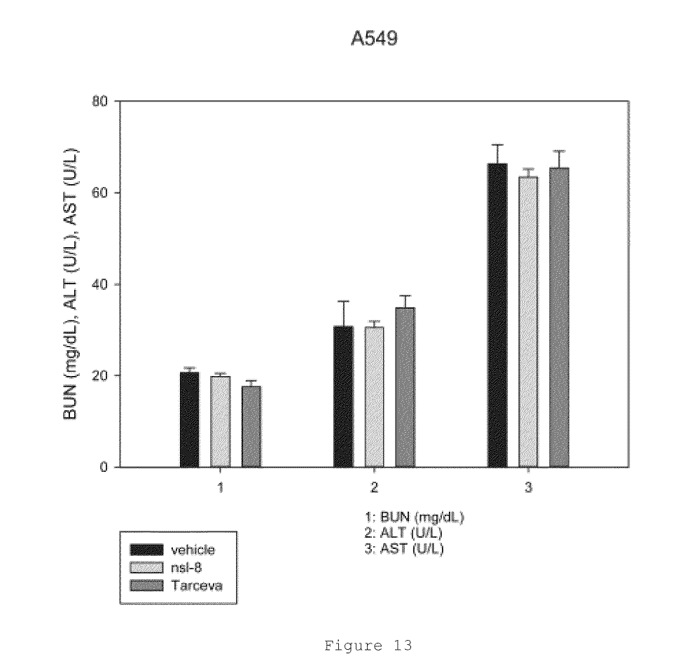

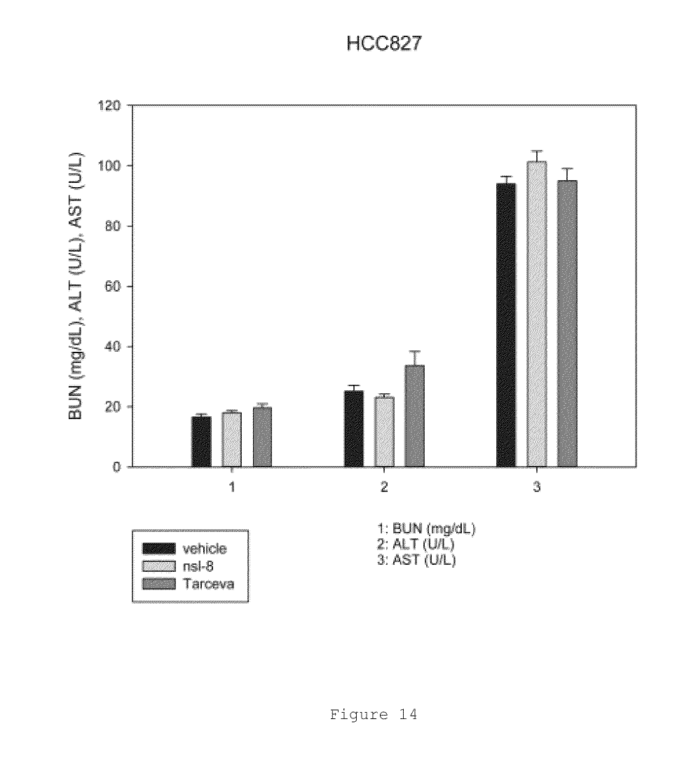

The complete blood count of peripheral blood samples collected from the A549 xenograft mice treated with nsl-8 and Tarceva® or Erlotinib was shown in FIG. 11. Apparently, there was no significant difference found in cell counts of white blood cell, red blood cell, or platelet among the vehicle, nsl-8, and Tarceva® or Erlotinib treated groups of the A549 model. However, there was an apparent difference in the complete blood count of peripheral blood samples collected from the HCC827 xenograft mice. As shown in FIG. 12, an increase in the counts of neutrophils and lymphocytes or decrease in the counts of red blood cells can be seen for the nsl-8 treated animals. There was also a decrease in red blood cells counted for the Tarceva® or Erlotinib treated animals of the same model (FIG. 12). However, there was no significant difference found in total white blood cells and platelets counted among all the vehicle and drugs treated groups of the same model (FIG. 12). Upon completion of the experiments, the serum levels of BUN, ALT, and AST were also determined. As shown in FIG. 13, there was no significant difference in serum levels of BUN, ALT, and AST determined among all the treatment groups of the A549 model. Similarly, there was also no significant difference in serum levels of BUN, ALT, and AST detected among all the treatment groups of the HCC827 model (FIG. 14). Therefore, these data would indicate that no apparent toxic effect was found in animals fed with 70 mg/kg nsl-8 daily during the entire experimental period.

We have found that there is no significant difference between the docking results of Tarceva® or Erlotinib by ADDock into either the wild type 2ity or double mutant enzyme 4i22. The drug is basically docked into the central catalytic cavity where ATP is usually bound (FIGS. 15a and 16a). However, there is an apparent difference between the docking results of nsl-8 by ADDock into either the wild type 2ity (FIG. 15b) or double mutant enzyme 4i22 (FIG. 16b). Moreover, the docking results of nsl-8 by ADDock in either wild type 2ity or double mutant enzyme 4i22 are quite different to those of Tarceva® or Erlotinib. The drug lead nsl-8 searched here may be useful for treating patients who have developed drug resistance after being treated with Tarceva® or Erlotinib. This is supported by the fact that the binding mode of nsl-8 with either wild type or mutant EGFR kinase domain (FIGS. 15b and 16b) is quite different to that of Tarceva® or Erlotinib (FIGS. 15a and 16a).

Based on both the in vivo monitoring of tumor growth as well as on the necropsy findings, a significant antitumoral efficacy with P=0.0838 could be found for nsl-8 administered at 40 mg/kg through i.p. route in the A549 tumor model. This efficacy is further confirmed in a second in vivo experiment for nsl-8 administered at 70 mg/kg through o.p. route in both the A549 and HCC827 tumor models. A 50% shrinkage in tumor size in the first A549 tumor model studied by injecting 40 mg/kg nsl-8 through i.p. route is observed, while a 28.7 or 19.1% shrinkage in tumor size in the HCC827 and second A549 tumor models studied by feeding 70 mg/kg nsl-8 through o.p. route is also observed. Based on the body weight monitored, blood cell counts determined, and serum levels of BUN, ALT, and AST measured, no apparent toxic effect is found for animals being treated with 70 mg/kg nsl-8 daily through o.p. route for 14 days. However, an apparent reduction in body weight is observed when the animals are treated with 40 mg/kg nsl-8 through i.p. route. We conjecture that such a decline may be caused by 10% DMSO used in preparing the dosage solutions, since the decline in body weight is observed in both nsl-8 and vehicle treated animals. It is known that EGFR has been targeted to treat breast^{14,15}, head and neck^{16,17}, pancreatic¹⁸, colorectal^{19,20}, glioma²¹⁻²³, esophageal^{24,25}, and gastrointestinal stromal^{26,27} cancers. Therefore, nsl-8 or adapalene which is effective in treating NSCLC confirmed here through studies on the two mouse xenograft models may be also effective for treating the aforementioned cancers.

All references, including but not limited to patents and publications, cited in this specification are herein incorporated by reference as though fully set forth.

Although the above specification accompanying with examples provided for the purpose of illustration, teaches the principles of the present invention, a person skilled in the art will be understood that the practice of the invention encompasses all of the usual variations, adaptations and/or modifications as come within the scope of the following claims and their equivalents.

### REFERENCES

1. Jemal, A., Siegel, R., Ward, E., Murray, T., Xu, J., Thun, M. J. Cancer Statistics. CA Cancer J. Clin. 2007, 57, 43-66.
2. Sharma, S. V., Bell, D. W., Settleman, J. and Haber, D. A. Epidermal growth factor receptor mutations in lung cancer. Nat. Rev. Cancer 2007, 7, 169-181.
3. Jackman, D., Pao, W., Riely, G. J., Engelman, J. A., Kris, M. G., Janne, P. A., Lynch, T., Johnson, B. E., Miller, V. A. Clinical definition of acquired resistance to epidermal growth factor receptor tyrosine kinase inhibitors in non-small-cell lung cancer. J. Clin. Oncol. 2010, 28, 357-360.
4. Zhou, W., Ercan, D., Chen, L., Yun, C. H., Li, D., Capelletti, M., Cortot, A. B., Chirieac, L., Iacob, R. E., Padera, R., et. al. Novel mutant-selective EGFR kinase inhibitors against EGFR T790M. Nature 2009, 462, 1070-1074.
5. Kim, Y., Li, Z., Apetri, M., Luo, B., Settleman, J. E., Anderson, K. S. Temporal resolution of autophosphorylation for normal and oncogenic forms of EGFR and differential effects of gefitinib. Biochemistry 2012, 51, 5212-5222.
6. Shan, Y., Eastwood, M. P., Zhang, X., Kim, E. T., Arkhipov, A., Dror, R. O., Jumper, J., Kuriyan, J., Shaw, D. E. Oncogenic mutations counteract intrinsic disorder in the EGFR kinase and promote receptor dimerization. Cell 2012, 149, 860-870.
7. Pao, W., Miller, V., Zakowski, M., Doherty, J., Politi, K., Sarkaria, I., Singh, B., Heelan, R., Rusch, V., Fulton, L. et al. EGF receptor gene mutations are common in lung cancers from “never smokers” and are associated with sensitivity of tumors to gefitinib and erlotinib. Proc. Natl. Acad. Sci. USA 2004, 101, 13306-13311.
8. Yun, C. H., Mengwasser, K. E., Toms, A. V., Woo, M. S., Greulich, H., Wong, K. K. Meyerson, M., Eck, M. J. The T790M mutation in EGFR kinase causes drug resistance by increasing the affinity for ATP. Proc. Natl. Acad. Sci. USA 2008, 105, 2070-2075.
9. Lin, T. H., Lin, G. L. An anchor-dependent molecular docking process for docking small flexible molecules into rigid protein receptors. J. Chem. Inf. Model. 2008, 48, 1638-1655.
10. Yun, C. H., Boggon, T. J., Li, Y., Woo, S., Greulich, H., Meyerson, M., Eck, N. J. Structures of lung cancer derived EGFR mutants and inhibitor complex: mechanism of activation and insights into differential inhibitor sensitivity. Cancer Cell 2007, 11, 217-227.
11. Gajiwala, K. S., Feng, J., Ferre, R. A., Ryan, K., Brodsky, O., Weinrich, S., Kath, J. C., Stewart, A. Insights into the aberrant activity of mutant EGFR kinase domain and drug recognition. Structure 2013, 21, 209-219.
12. Ono, M., Hirata, A., Kometani, T., Miyagawa, M., Ueda, S. I. Kinoshita, H., Fujii, T., Kuwano, M. Sensitivity to gefitinib (Iressa, ZD1839) in non-small cell lung cancer cell lines correlates with dependence on the epidermal growth factor (EGF) receptor/Akt pathway for proliferation. Mol. Cancer Ther. 2004, 3, 465-472.
13. Li, T., Ling, Y. H., Goldman, D., Perez-Soler, R. Schedule-dependent cytotoxic synergism of pemetrexed and erlotinib in human non-small cell lung cancer cells. Clin. Cancer Res. 2007, 13, 3413-3422.
14. Chen, P., Cameron, R., Wang, J., Wallis, K. A., Reilly, R. M. Antitumor effects and normal tissue toxicity of 111In-labeled epidermal growth factor administered to athymic mice bearing epidermal growth factor receptor-positive human breats cancer xenografts. J. Nucl. Med. 2003, 44, 1469-1478.
15. Natha, R., Trent, S., Yang, C., Schmidt, E. V. Epidermal growth factor expression is a candidate target of the synergistic combination of trastuzumab and flavopiridol in breast cancer. Cancer Res. 2003, 63, 3626-3631.
16. Bonner, J. A., De Los Santos, J., Waksal, H. W., Needle, M. N., Trummel, H. Q., Raisch, K. P. Epidermal growth factor receptor as a therapeutic target in head and neck cancer. Semin. Oncol. 2002, 12(3 Suppl 2), 11-20.
17. Epidermal growth factor receptors as a target for cancer treatment: the emerging role of IMC-C225 in the treatment of lung and head and neck cancers. Semin. Oncol. 2002, 29(1 Suppl 4), 27-36.
18. Xiong, H. Q., Abbruzzese, J. L. Epidermal growth factor receptor-targeted therapy for pancreatic cancer. Semin. Oncol. 2002, 29(5 Suppl 14), 31-37.
19. Gravalos, C., Cassinello, J., Garcia-Alfonso, P., Jimeno, A. Integration of panitumumab into the treatment of colorectal cancer. Crit. Rev. Oncol. Hematol. 2010, 74, 16-26.
20. Mano, M., Humblet, Y. Drug Insight: panitumumab, a human EGFR-targeted monoclonal antibody with promising clinical activity in colorectal cancer. Nat. Clin. Pract. Oncol. 2008, 5, 415-425.
21. Alqudah, M. A., Agarwal, S., Al-Keilani, M. S., Sibenaller, Z. A., Ryken, T. C., Assem, M. NOTCH3 is a prognostic factor that promotes glioma cell proliferation, migration, and invasion via activation of CCND1 and EGFR. PLoS One 2013, 8, e77299.
22. Burel-Vandenbos, F., Turchi, L., Benchetrit, M., Fontas, E., Pedeutour, Z., Rigau, V., Almairac, F., Ambrosetti, D., Michiels, J. F., Virolle, T. Cells with intense EGFR staining and a high nuclear to cytoplasmic neuropathological practice. Neuro. Oncol. 2013, 15, 1278-1288.
23. Schulte, A., Liffers, K., Kathagen, A., Riethdorf, S., Zapf, S., Merlo, A., Kolbe, K., Westphal, M., Lamszus, K. Erlotinib resistance in EGFR-amplified glioblastoma cells is associated with upregulation of EGFRvIII and P13Kp110o. Neuro. Onclo. 2013, 15, 1289-1301.
24. Drenckhan, A., Grob, T., Dupree, A., Dohrmann, T., Mann, O., Izbicki, J. R. Esophageal carcinoma cell line with high EGFR polysomy is responsive to gefitinib. Langenbecks Arch. Surg. 2014, 399, 879-888.
25. Wang, J., Yu, J. M., Jing, S. W., Guo, Y., Wu, Y. J., Li, N., Jiao, W. P., Wang, L., Zhang, Y. J. Relationship between EGFR over-expression and clinicopathologic characteristics in squamous cell carcinoma of the esophagus: A meta-analysis. Asian Pac. J. Cancer Prey. 2014, 15, 5889-5893.
26. Cervera, P., Flejou, J. F. Changing pathology with changing drugs: tumors of the gastrointestinal tract. Pathobiology 2011, 78, 76-89.
27. Nagata, K., Kawakami, T., Kurata, Y., Kimura, Y., Suzuki, Y., Nagata, T., Sakuma, Y., Miyagi, Y., Hirano, H. Augmentation of multiple protein kinase activities associated with secondary imatinib resistance in gastrointestinal stromal tumors as revealed by quantitative phosphoproteome analysis. J. Proteomics 2015, 115, 132-142.

What is claimed is:

1. A method of treating a cancer in a subject comprising administering to the subject 6-[3-(1-adamantyl)-4-methoxyphenyl]-2-naphthalenecarboxylic acid or a pharmaceutical salt or derivative thereof in need of such treatment, wherein the cancer is esophageal cancer or gastrointestinal stromal tumor.

2. The method of claim 1, wherein the subject in need of such treatment is a mammal.

3. The method of claim 2, wherein the mammal is human.

4. The method claim 1, wherein the cancer is esophageal cancer.

5. The method claim 1, wherein the cancer is gastrointestinal stromal tumor.

6. The method of claim 1, wherein the 6-[3-(1-adamantyl)-4-methoxyphenyl]-2-naphthalenecarboxylic acid or a pharmaceutical salt or derivative thereof is administrated to the subject at a daily dosage which is converted from a mouse daily dosage of about 40 mg/kg body weight.

7. The method of claim 1, wherein the 6-[3-(1-adamantyl)-4-methoxyphenyl]-2-naphthalenecarboxylic acid is administered to the subject in a solution form via intraperitoneal injection route.

8. The method of claim 7, wherein the solution is prepared by dissolving 6-[3-(1-adamantyl)-4-methoxyphenyl]-2-naphthalenecarboxylic acid in an organic solvent and mixing the organic solution of the 6-[3-(1-adamantyl)-4-methoxyphenyl]-2-naphthalenecarboxylic acid with an aqueous solution.

9. A method of treating a cancer in a subject comprising administering to the subject an agent active in binding with EGFR kinase domain in the subject in need of such treatment, wherein the cancer is esophageal cancer, and the agent comprises 6-[3-(1-adamantyl)-4-methoxyphenyl]-2-naphthalenecarboxylic acid or a pharmaceutical salt or derivative thereof.

10. A method of treating a cancer in a subject comprising administering to the subject an agent active in binding with EGFR kinase domain in the subject in need of such treatment, wherein the cancer is gastrointestinal stromal tumor, and the agent comprises 6-[3-(1-adamantyl)-4-methoxyphenyl]-2-naphthalenecarboxylic acid or a pharmaceutical salt or derivative thereof.
